(19) United States
(12) Patent Application Publication
(10) Pub. No.: US 2019/0357799 A1
(43) Pub. Date: Nov. 28, 2019

(54) MAGNETIC RESONANCE IMAGING APPARATUS AND METHOD OF CONTROLLING THE SAME

(71) Applicant: SAMSUNG ELECTRONICS CO., LTD., Suwon-si (KR)

(72) Inventors: Sang-Cheon CHOI, Suwon-si (KR); Dae Ho LEE, Seongnam-si (KR)

(73) Assignee: SAMSUNG ELECTRONICS CO., LTD., Suwon-si (KR)

(21) Appl. No.: 16/461,712

(22) PCT Filed: Oct. 17, 2017
(86) PCT No.: PCT/KR2017/011463

§ 371 Date: May 16, 2019

(30) Foreign Application Priority Data
Nov. 16, 2016 (KR) 10-2016-0152408

(51) Int. Cl. A61B 5/055; G01R 33/483; G01R 33/24; G01R 33/385

(52) U.S. Cl. CPC A61B 5/055; G01R 33/4835; G01R 33/543; G01R 33/385; G01R 33/24

(57) ABSTRACT

A magnetic resonance imaging (MRI) apparatus and a method of controlling the same is disclosed. The MRI apparatus includes a magnetic field generator configured to apply a magnetic field to a head of a subject; a radio frequency (RF) coil configured to apply a pulse to the head to which the magnetic field is applied, and to receive a signal generated in the head; and a processor configured to apply a first inversion recovery pulse to the head by the RF coil, when a magnitude of longitudinal magnetization of one of white matter (WM) and gray matter (GM) of the head is in a first range, suppress a recovery signal corresponding to the longitudinal magnetization of at least one of the WM and GM among recovery signals generated according to the first inversion recovery pulse; and generate a cerebrospinal fluid (CSF) image for at least one slice based on a signal at a point where a magnitude of transverse magnetization generated in the other of the WM and GM of the head is in a second range.

### TECHNICAL FIELD

The disclosure relates to a magnetic resonance imaging apparatus and a method of controlling the same.

### BACKGROUND ART

An image capturing apparatus refers to an apparatus for obtaining external or internal information of an object using visible rays, infrared rays, ultrasound, radioactive rays, Nuclear Magnetic Resonance (NMR) or the like, and providing images of the obtained information to a user.

Examples of the image capturing apparatus may include a camera, an ultrasonic imaging apparatus, a digital radiographic apparatus, a computed tomographic (CT) apparatus, a mammographic apparatus, or a magnetic resonance imaging (MRI) apparatus. Various conductive lines or circuits may be used to transmit electrical signals in the above-mentioned image capturing apparatuses, for example, a crossbar switch matrix may be used.

The MRI apparatus refers to an apparatus for obtaining a cross-sectional image of the inside of a subject such as a human, an animal, or a plant using the phenomenon of the NMR.

The MRI apparatus may obtain MRI signals, applying a gradient to magnetic resonance signals induced by a magnetization vector of an atomic nucleus exposed to a magnetic field to a nearby RF coil, i.e., free induction decay (FID) signals, and obtain MRI images using the obtained MRI signals.

### DISCLOSURE

### Technical Problem

Therefore, it is an aspect of the disclosure to provide a magnetic resonance imaging (MRI) apparatus capable of obtaining a plurality of flair images (FLAIR images) or a plurality of T2-weighted images in a relatively short period of time, and a method of controlling the same.

It is another aspect of the disclosure to provide a magnetic resonance imaging (MRI) apparatus capable of obtaining clearer and clear final images using the obtained flair images (FLAIR images) or T2-weighted images, and a method of controlling the same.

### Technical Solution

In order to solve the above-described problems, the disclosure is to provide a magnetic resonance imaging apparatus and a method of controlling the same.

One aspect of the disclosure provides a magnetic resonance imaging (MRI) apparatus including: a magnetic field generator configured to apply a magnetic field to a head of a subject; a radio frequency (RF) coil configured to apply a pulse to the head to which the magnetic field is applied, and to receive a signal generated in the head; and a processor configured to apply a first inversion recovery pulse to the head by the RF coil, when the magnitude of the longitudinal magnetization of either white matter (WM) and gray matter (GM) of the head is in a first range, suppress a recovery signal corresponding to the longitudinal magnetization of at least one of the WM and GM among recovery signals generated according to the first inversion recovery pulse; and generate a cerebrospinal fluid (CSF) image for at least one slice based on a signal at a point where the magnitude of the transverse magnetization generated in the other of the WM and GM of the head is in a second range.

When the RF coil applies a second inversion recovery pulse to the head and the magnitude of the longitudinal magnetization of the CSF of the head is in a third range, the processor may suppress a recovery signal generated in the CSF according to the second inversion recovery pulse to obtain a flair image for a slice different from the at least one slice.

The RF coil may apply the second inversion recovery pulse to the head, and apply the first inversion recovery pulse to the head before the flair image for the slice different from the at least one slice is obtained.

The RF coil may apply the pulse to the head, and the processor may generate a second T2-weighted image for the at least one slice based on a magnetic resonance signal received by the RF coil.

The RF coil may apply an inversion recovery pulse to the head, and the processor may suppress a recovery signal generated in the CSF of the head to obtain a first flair image for the at least one slice.

The processor may combine the first flair image and the CSF image for the at least one slice to obtain a first T2-weighted image for the at least one slice.

The processor may combine the first T2-weighted image and the second T2-weighted image by performing weighted summing, square summing, or complex summing of the first T2-weighted image and the second T2-weighted image to obtain a final image.

The processor may obtain a second flair image for the slice by subtracting the CSF image from the second T2-weighted image.

The processor may obtain a final image based on a first flair image and the second flair image.

The processor may obtain the magnetic resonance signal using a multiband radio frequency pulse method or obtain the magnetic resonance signal using an interleaved acquisition method.

The first range may include the magnitude of the longitudinal magnetization of either the WM or GM of the head at a value of 0 or close thereto, and the second range may include the magnitude of the transverse magnetization of the other of the WM and GM of the head at a value of 0 or close thereto.

Another aspect of the disclosure provides a method of controlling a magnetic resonance imaging (MRI) apparatus, including: applying, by a magnetic field generator, a magnetic field to a head of a subject; applying, by an RF coil, a first inversion recovery pulse to the head to which the magnetic field is applied when the magnitude of the longitudinal magnetization of either white matter (WM) and gray matter (GM) of the head is in a first range; suppressing, by a processor, a recovery signal corresponding to the longitudinal magnetization of at least one of the WM and GM among recovery signals generated according to the first inversion recovery pulse; and generating, by the processor, a cerebrospinal fluid (CSF) image for at least one slice based on a signal at a point where the magnitude of the transverse magnetization generated in the other of the WM and GM of the head is in a second range.

The method may further include applying, by the RF coil, a second inversion recovery pulse to the head to which the magnetic field is applied; and when the magnitude of the longitudinal magnetization of the CSF of the head is in a third range, suppressing, by the processor, a recovery signal generated in the CSF according to the second inversion recovery pulse to obtain a flair image for a slice different from the at least one slice.

The applying of the first inversion recovery pulse to the head to which the magnetic field is applied may be performed after the applying of the second inversion recovery pulse to the head to which the magnetic field is applied.

The method may further include obtaining, by the processor, a flair image for the at least one slice; and combining, by the processor, the flair image for the at least one slice and the CSF image for the at least one slice to obtain a first T2-weighted image for the at least one slice.

The method may further include receiving, by the RF coil, a magnetic resonance signal generated in the head; generating, by the processor, a second T2-weighted image for the at least one slice based on the magnetic resonance signal; and obtaining a final image based on the first T2-weighted image and the second T2-weighted image.

The obtaining of the final image based on the first T2-weighted image and the second T2-weighted image may include combining the first T2-weighted image and the second T2-weighted image by performing weighted summing, square summing, or complex summing of the first T2-weighted image and the second T2-weighted image to obtain the final image.

The method may further include receiving, by the RF coil, a magnetic resonance signal generated in the head; generating, by the processor, a T2-weighted image for the at least one slice based on the magnetic resonance signal; and obtaining, by the processor, a second flair image for the at least one slice by subtracting the CSF image from the T2-weighted image.

The method may further include applying, by the RF coil, an inversion recovery pulse to the head to which the magnetic field is applied; suppressing, by the processor, a recovery signal generated in the CSF of the head to obtain a first flair image for the at least one slice; and obtaining, by the processor, the final image based on the first flair image and the second flair image.

The first range may include the magnitude of the longitudinal magnetization of either the WM or GM of the head at a value of 0 or close thereto, and the second range may include the magnitude of the transverse magnetization of the other of the WM and GM of the head at a value of 0 or close thereto.

### Advantageous Effects

According to the above-described MRI apparatus and the method of controlling the same, the plurality of flair images (FLAIR images) or the plurality of T2-weighted images can be obtained more quickly, and an effect in which a capturing time for obtaining a plurality of images is relatively shortened can be obtained.

According to the above-described MRI apparatus and the method of controlling the same, it may be possible to obtain clearer and more definite final images by using the obtained flair images or T2-weighted images, thereby allowing a user such as a doctor to more accurately grasp and diagnose the structure inside a subject.

In addition, according to the above-described MRI apparatus and the method of controlling the same, a plurality of inversion recovery signals (IR signals) are applied to the subject in a single concatenation, thereby obtaining a flair image for a first slice and a cerebrospinal fluid image (CSF image) for a second slice in the concatenation.

### DESCRIPTION OF THE DRAWINGS

FIG. 1 is a view illustrating a magnetic resonance imaging (MRI) apparatus according to an embodiment.

FIG. 2 is a graph illustrating an example of an inversion recovery curve according to an embodiment.

FIG. 3 is a graph illustrating an example of a T2 decay curve according to an embodiment.

FIG. 4 is a graph illustrating an inversion recovery curve and a T2 decay curve of each configuration of a brain to explain a process of obtaining a flair (FLAIR) image according to an embodiment.

FIG. 5 is a view illustrating an example of the obtained flair image according to an embodiment.

FIG. 6 is a graph illustrating the T2 decay curve of each configuration of the brain to explain a process of obtaining a T2-weighted image according to an embodiment.

FIG. 7 is a view illustrating an example of the obtained T2-weighted image according to an embodiment.

FIG. 8 is a view illustrating an inversion recovery curve and a T2 decay curve of each configuration of the brain to explain a process of obtaining a cerebrospinal fluid image (CSF image) according to an embodiment.

FIG. 9 is a view illustrating an example of the obtained CSF image according to an embodiment.

FIG. 10 is a view illustrating image acquisition in a first scan and a second scan according to an embodiment.

FIG. 11 is a view illustrating a first slice and a second slice according to an embodiment.

FIG. 12 is a view illustrating an example of obtaining a new T2-weighted image according to a combination of the flair image and a CSF-only image according to an embodiment.

FIG. 13 is a view illustrating an example of obtaining a new flair image according to subtraction of the CSF-only image in the T2-weighted image according to an embodiment.

FIG. 14 is a first flowchart of a method of controlling the MRI apparatus according to an embodiment.

FIG. 15 is a second flowchart of a method of controlling the MRI apparatus according to an embodiment.

FIG. 16 is a third flowchart of a method of controlling the MRI apparatus according to an embodiment.

### MODE OF THE INVENTION

Like reference numerals refer to like elements throughout the specification. Not all elements of embodiments of the disclosure will be described, and description of what are commonly known in the art or what overlap each other in the embodiments will be omitted. The terms as used throughout the specification, such as “˜part,” “˜module,” “˜member,” “˜block,” etc., may be implemented in software and/or hardware, and a plurality of “˜parts,” “˜modules,” “˜members,” or “˜blocks” may be implemented in a single element, or a single “˜part,” “˜module,” “˜member,” or “˜block” may include a plurality of elements.

It will be understood that when an element is referred to as being “connected” to another element, it can be directly or indirectly connected to the other element, wherein the indirect connection includes “connection through a wireless communication network.”

Also, when a part “includes” or “comprises” an element, unless there is a particular description contrary thereto, the part may further include other elements, not excluding the other elements.

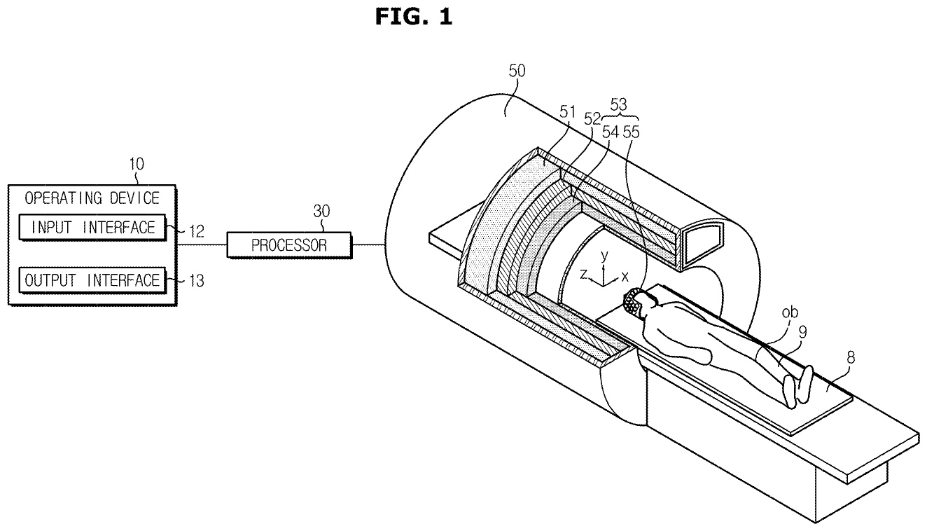

Hereinafter, exemplary embodiments of a magnetic resonance imaging (MRI) apparatus will be described with reference to FIGS. 1 to 13.

FIG. 1 is a view illustrating a magnetic resonance imaging (MRI) apparatus according to an embodiment.

Referring to FIG. 1, an MRI apparatus 1 may include an operating device 10, a processor 30, and a scanner 50. The operating device 10, the processor 30, and the scanner 50 are provided to communicate with each other through at least one of wired communication technology and wireless communication technology. Here, the wired communication technology may be implemented using various cables such as pair cables, coaxial cables, fiber optic cables, and Ethernet cables. The wireless communication technology may be implemented using at least one of short range communication technology and long-distance communication technology. The short range communication technology may include wireless fidelity (Wi-Fi), ZigBee, Bluetooth, Wi-Fi Direct, Bluetooth Low Energy, Near Field Communication (NFC), or the like. Further, the long-distance communication technology may include various communication technologies based on various mobile communication standards such as 3rd Generation Partnership Project (3GPP), 3GPP2, Worldwide Interoperability for Microwave Access (WiMAX) series, or the like.

The operating device 10 may receive various commands used to operate the MRI apparatus 1 from a user or may display an image corresponding to an electrical signal obtained through the scanner 50 to the user.

The operating device 10 may include at least one of an input interface 12 and an output interface 13.

The input interface 12 may receive a control command related to the overall operation of the MRI apparatus 1 from the user. For example, the input interface 12 may receive subject information, parameter information, scan conditions, information on pulse sequences, or the like, from the user. The input interface 12 may be implemented as, for example, each of a physical button such as a keyboard and the like, a mouse, a stick manipulation device, a trackball, a voice recognition device, a gesture recognition device, a touch screen, or the like, or may be implemented as a combination thereof.

The output interface 13 may output at least one image and provide the image to the user. In addition, the output interface 13 may be implemented using a display device for displaying an image or may be implemented using a speaker. The output interface 13 may also output a graphic user interface (GUI) configured to allow the user to input a control command related to the MRI apparatus 1. The output interface 13 may be implemented, for example, using a cathode ray tube (CRT) or various other types of display panels such as a liquid crystal display (LCD) panel, a light-emitting diode (LED) panel, an organic light-emitting diode (OLED) panel, and the like.

The processor 30 may generate a control signal related to the overall operation of the MRI apparatus 1 according to a control command of the user that is input through the input interface 12, a predefined setting, or a program, transfer the generated control signal to each of the components, and allow the MRI apparatus 1 to perform a predetermined operation.

For example, the processor 30 may control a static magnetic field generator 51 or a gradient magnetic field generator 52 to receive predetermined power so that the static magnetic field generator 51 or the gradient magnetic field generator 52 may apply a static magnetic field or a gradient magnetic field to a subject 9. The processor 30 may transmit the control signal to a radio frequency (RF) coil 53 of the scanner 50 and allow the RF coil 53 to transmit an RF pulse to the subject 9. The processor 30 may also control the RF coil 53 to apply an inversion recovery pulse, i.e., a 180 degree RF pulse, to the subject 9 so that the RF coil 53 obtains a recovery signal from the subject 9. In addition, the processor 30 may control the MRI apparatus 1 to perform various operations.

Further, the processor 30 may allow at least one memory to store a magnetic resonance signal obtained by the scanner 50, and may generate an image of the subject 9 using the stored magnetic resonance signal.

For example, the processor 30 may obtain k-space data based on the magnetic resonance signal, generate a k-space by arranging the obtained k-space data according to the predefined definition, and obtain the image of the subject 9 from the generated k-space by applying various restoration methods such as a Fourier transform and the like. The processor 30 may amplify the magnetic resonance signal and/or convert the magnetic resonance signal into a digital signal before the k-space data is obtained.

The processor 30 may perform various signal processes applied to the magnetic resonance signal in parallel. For example, the processor 30 may obtain an image by processing a plurality of magnetic resonance signals transmitted from a multi-channel RF coil part in parallel.

Further, the processor 30 may further perform post image processing such as emphasizing the contrast of the acquired magnetic resonance image or removing noise therefrom, or may generate a new image by combining a plurality of images or by subtracting another image from one image. These operations will be described below.

The processor 30 may be implemented using at least one semiconductor chip and related parts. The processor 30 may include, for example, a central processing unit (CPU), a micro controller unit (MCU), a micro-processor unit (MPU), or the like.

In some exemplary embodiments, the operating device 10 and the processor 30 may be implemented as separate devices, as illustrated in FIG. 1, or may be physically included in one device. Further, in some exemplary embodiments, at least one of the operating device 10 and the processor 30 may be embedded in an external housing of the scanner 50.

The scanner 50 is provided to obtain the magnetic resonance signal from the subject 9.

An internal room 50a which is empty so that the subject 9 is inserted therein, for example, a bore, is provided in the scanner 50. The scanner 50 may include a transfer device 8 for transferring the subject 9 to an internal space, the static magnetic field generator 51 for forming a static magnetic field in the subject 9 inserted into the internal room 50a, the gradient magnetic field generator 52 for forming a gradient magnetic field in the subject 9 inserted into the internal room 50a, and the RF coil 53 for applying the RF pulse to the subject 9 and receiving the magnetic resonance signal generated by the subject 9.

The transfer device 8 may have a shape of a table and may be moved into or out of the internal space in accordance with the control signal of the processor 30, thereby inserting or removing the subject 9 into or from the internal space.

The static magnetic field generator 51 is formed around the internal space and is provided to generate a static magnetic field in the internal room 50a. The generated static magnetic field magnetizes atoms which cause a magnetic resonance phenomenon among elements distributed in the subject 9 (e.g., a human body) for example, an atomic nucleus of an element such as hydrogen, phosphorus, sodium, or the like. The static magnetic field generator 51 may be made of a superconducting electromagnet or a permanent magnet. In order to generate a magnetic field having a high magnetic flux density of 0.5 Tesla or more, a superconducting electromagnet is used as the static magnetic field generator 51.

The gradient magnetic field generator 52 is provided to generate spatially linear gradient magnetic fields Gx, Gy, and Gz in the subject 9 and to induce a change in the uniformity of the magnetic field. The gradient magnetic field generator 52 may include X coils, Y coils, and Z coils forming mutually perpendicular gradient magnetic fields in the X-axis, Y-axis, and Z-axis directions, and may generate a gradient signal corresponding to a capturing position so as to induce resonance frequencies of the respective parts of the subject 9 different from each other. As the magnetization vector of the atomic nucleus generated by the static magnetic field rotates on a transverse plane, the rotational frequency and phase of the magnetization vector become spatially controllable by the gradient magnetic field. Thus, the signal obtained by the RF coil 53 can be expressed as a spatial frequency domain, i.e., the k-space.

The RF coil 53 may irradiate the RF pulse to the subject 9 in accordance with the control signal received from the processor 30 and receive the magnetic resonance signal emitted from the subject 9. The RF coil 53 may transmit an RF signal having the same frequency as a frequency of procession toward the atomic nucleus which performs precession to the subject 9, and the RF coil 53 may receive the magnetic resonance signal emitted from the subject 9 in accordance with the interruption of the RF signal.

According to an exemplary embodiment, the RF coil 53 may separately include a transmitting RF coil for generating an electromagnetic wave (i.e., an RF pulse) having a radio frequency corresponding to a type of the atomic nucleus, and a receiving RF coil for receiving an electromagnetic wave (i.e., a magnetic resonance signal) emitted from the atomic nucleus. According to another exemplary embodiment, the RF coil 53 may include an RF transmitting and receiving coil which may perform both functions of transmitting the RF pulse and receiving the magnetic resonance signal.

According to an exemplary embodiment, the RF coil 53 may be mounted on all or part of a body coil 54 and/or the subject 9 provided inside the MRI apparatus 1, as illustrated in FIG. 1. The RF coil 53 mounted on the subject 9 may also include a head coil, a spine coil, a torso coil, and/or a knee coil according to an imaging part.

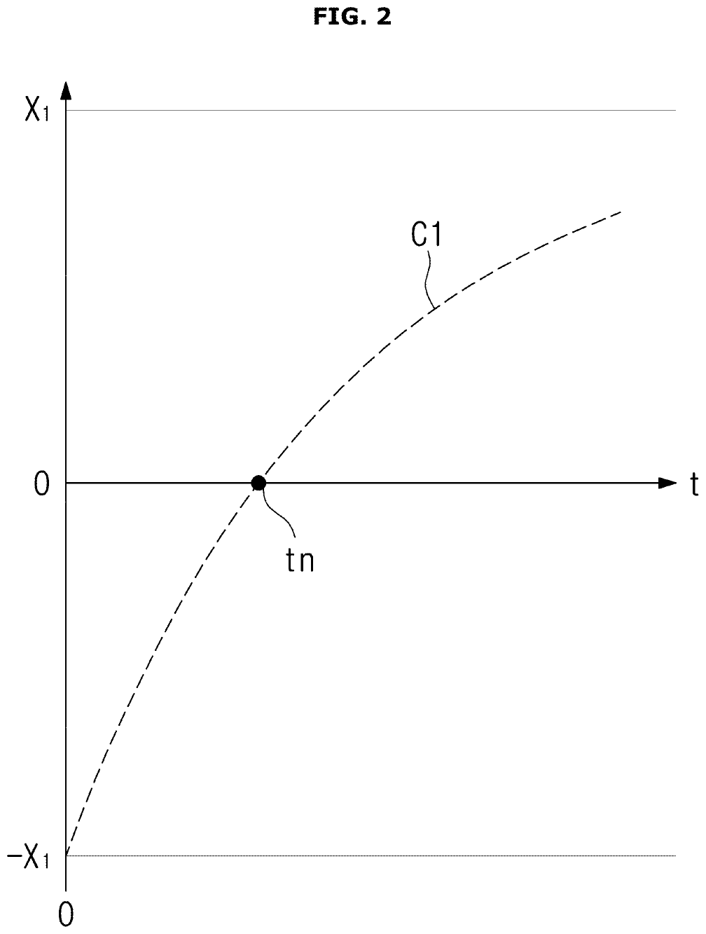

FIG. 2 is a graph illustrating an example of an inversion recovery curve. In FIG. 2, an X-axis denotes a time and a Y-axis denotes a magnitude of magnetization or intensity of a signal.

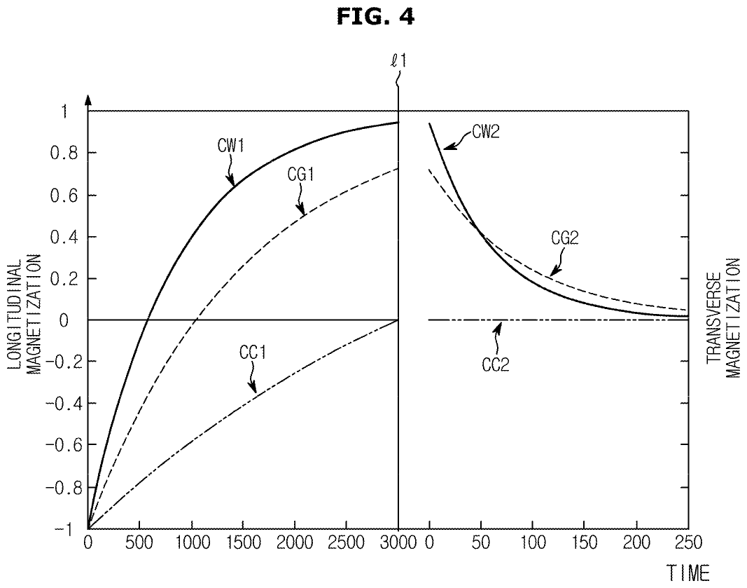

As described above, when the RF coil 53 irradiates the subject 9 with the RF pulse and then stops irradiating the RF pulse, spin of the atomic nucleus is aligned in a direction of the static magnetic field while emitting the supplied energy. As a result, the longitudinal magnetization of the atomic nucleus is gradually recovered to the initial magnetization, and the recovery signal obtained also corresponds to the recovery of the longitudinal magnetization. In addition, the RF coil 53 may apply the inversion recovery pulse, i.e., the 180 degree RF pulse, to the subject 9. When the inversion recovery pulse is applied to the subject 9 when the static magnetic field and the gradient magnetic field are applied to the subject 9, the longitudinal magnetization of the atomic nucleus of the subject 9 is formed in the opposite direction and has a negative value −x1 as illustrated in FIG. 2. When the RF coil 53 stops applying the inversion recovery pulse, the longitudinal magnetization of the atomic nucleus gradually recovers from the negative value −x1 along a recovery curve c1, and finally approaches x1. In this case, the recovery curve c1 of the longitudinal magnetization is different for each substance to which the inversion recovery pulse is applied. For example, the longitudinal magnetization in white matter (WM), gray matter (GM), and CSF of the brain are recovered along different curves CW1, CG1, CC1 in FIG. 4. During a recovery process, the longitudinal magnetization passes through a zero point of the graph at a certain time point tN, where the zero point of longitudinal magnetization is called a null point. The strength of the recovery signal obtained at the null point is zero.

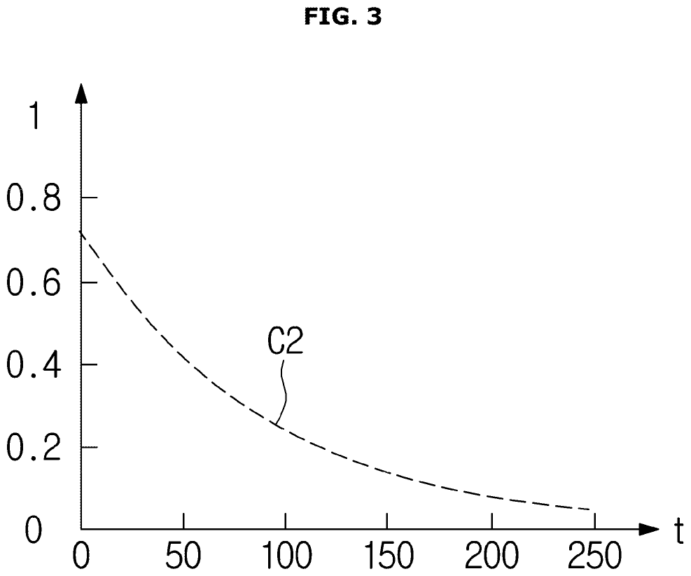

FIG. 3 is a graph illustrating an example of a T2 decay curve. In FIG. 3, an X-axis denotes a time and a Y-axis denotes a magnitude of magnetization or intensity of a signal.

When the RF coil 53 applies the RF pulse to the subject 9 when the static magnetic field and the gradient magnetic field are applied to the subject 9, the atomic nucleus, i.e., a proton, performs precession according to a new magnetic component of the RF pulse. In other words, the transverse magnetization vector may be generated in the atomic nucleus. If the RF pulse of the same frequency is applied to the atomic nucleus, which performs precession at a predetermined frequency, an electrical signal having an electromotive force of a predetermined magnitude, i.e., a free induction decay (FID) signal, is induced in the RF coil 53 by the rotation of the magnetization vector, i.e., spin. If the application of the RF pulse stops, the transverse magnetization vector may decay (T2 decay) along a predetermined curve c2 as illustrated in FIG. 3. As a result, the obtained signal may also attenuate with the progress of time. The decay of such transverse magnetization is different for each substance. Therefore, the intensity of the electrical signal obtained from each substance changes according to a set echo delay time (TE), and thus various images may be obtained.

Hereinafter, the process of obtaining the flair image will be described as an example of a case where the subject is a head of a human body.

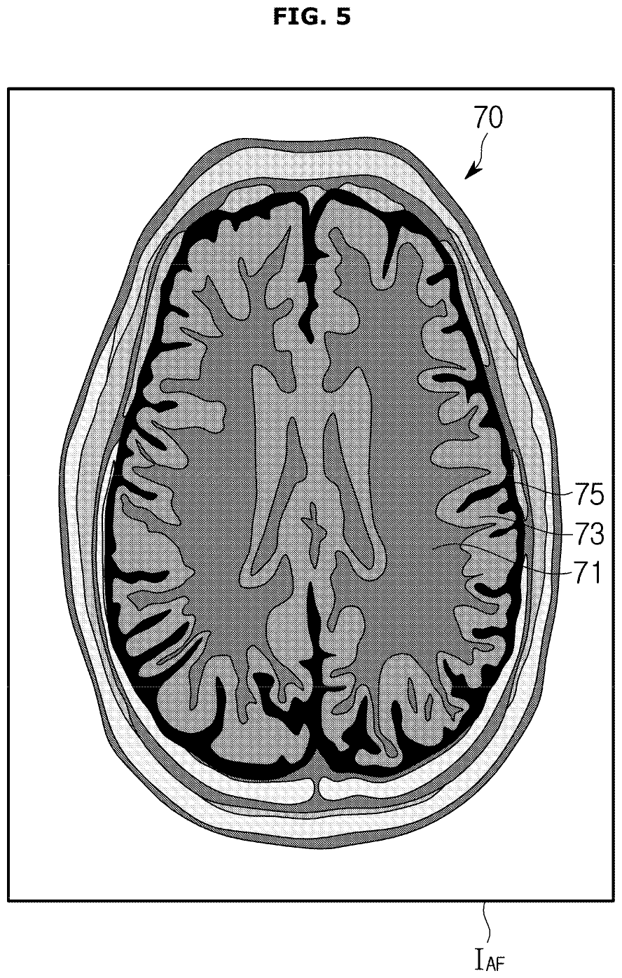

FIG. 4 is a graph illustrating an inversion recovery curve and a T2 decay curve of each configuration of a brain to explain a process of obtaining a flair image, and FIG. 5 is a view illustrating an example of the obtained flair image.

In FIG. 4, the left side of a middle vertical line (11) illustrates the time-dependent recovery of the longitudinal magnetization of each substance with the application of the inversion recovery pulse, and the right side of the vertical line (11) illustrates the time-dependent decay of the transverse magnetization. On the left side of FIG. 4, CW1 illustrates recovery of the longitudinal magnetization of the WM in the form of a graph, CG1 illustrates recovery of the longitudinal magnetization of the GM, and CC1 illustrates recovery of the longitudinal magnetization of the CSF. CW2 illustrates decay of the transverse magnetization of the WM, CG2 illustrates decay of the transverse magnetization of the GM, and CC2 illustrates decay of the transverse magnetization of the CSF. FIGS. 4 and 5 illustrate graphs and magnetic resonance images, for example, when a magnetic field of Tesla 3.0 is applied to the subject 9, but the magnetic field applied to the subject 9 is not limited thereto.

When the inversion recovery pulse is applied to the head, the longitudinal magnetization of protons such as hydrogen atoms or water molecules constituting WM, GM, and CSF is formed in the opposite direction. When the inversion recovery pulse is stopped, the longitudinal magnetization gradually recovers as time elapses, as illustrated in FIG. 5 (CW1, CG1, CC1). Since the longitudinal magnetization of the CSF is relatively slower than the longitudinal magnetization of other substances (CC1), the longitudinal magnetization of the CSF is almost recovered when the longitudinal magnetization reaches 0 or close thereto.

When the longitudinal magnetization of the CSF reaches a predetermined range, the processor 30 may excite the atomic nucleus by transmitting an excitation pulse to the atomic nucleus so that the electrical signal generated in the CSF, i.e., the recovery signal, become 0 or a very small value. In other words, according to the inversion recovery pulse, the recovery signal from the subject is suppressed.

According to an exemplary embodiment, the predetermined range may be defined to include only the case where the magnitude of the longitudinal magnetization of the CSF is 0, and the magnitude of the longitudinal magnetization of the CSF may be defined as a range around 0, for example, at least one value (e.g., 0+/−a). For example, when the predetermined range is defined as 0, excitation to the atomic nucleus is performed at the position where the vertical line (11) of FIG. 4 is located. The predetermined range may be set by the user, or may be defined in advance by the designer.

Thus, when the recovery signal generated in the CSF is maintained at a value of 0 or close thereto, the image may be obtained using only electrical signals corresponding to the longitudinal magnetization of WM and GM.

Particularly, as illustrated on the right side of FIG. 4, the transverse magnetization of the CSF maintains a value of 0 or close thereto (CC2), and the transverse magnetization of the WM and the transverse magnetization of the GM are gradually decayed (CW2, CG2).

The processor 30 may generate a T2-weighted image based on the electrical signal corresponding to the longitudinal magnetization of WM and GM at a certain time point. In this case, the processor 30 may generate the T2-weighted image using a fast spinning echo (FSE) manner. As a result, as illustrated in FIG. 5, the CSF is removed (CSF Nulling) and an image IAF is obtained for a head 70 where WM and GM are mainly expressed. The image from which the obtained CSF is removed is called the flair image IAF. In the flair image IAF, a part 71 where the WM exists and a part 73 where the GM exists illustrate relatively bright colors, but a part 75 where the CSF exists is expressed in black because the signal is not fully or hardly obtained.

Hereinafter, a process of obtaining the T2-weighted image of the head of the human body will be described as an example.

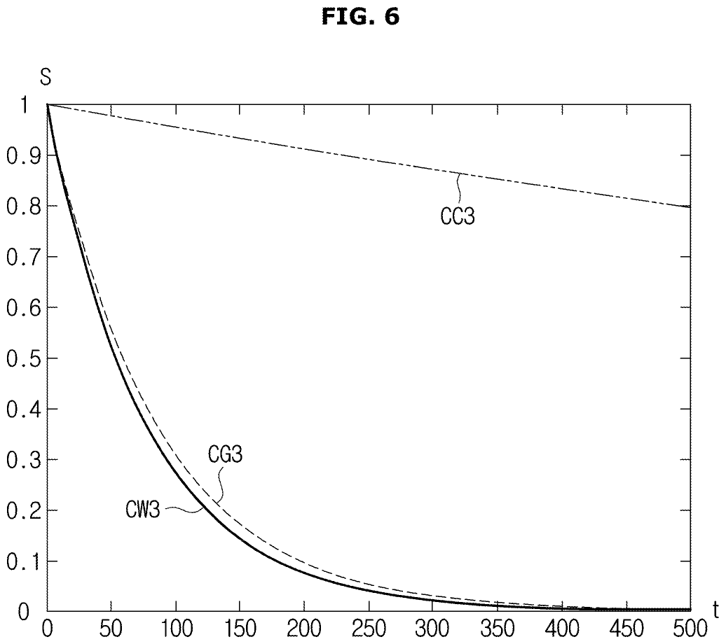

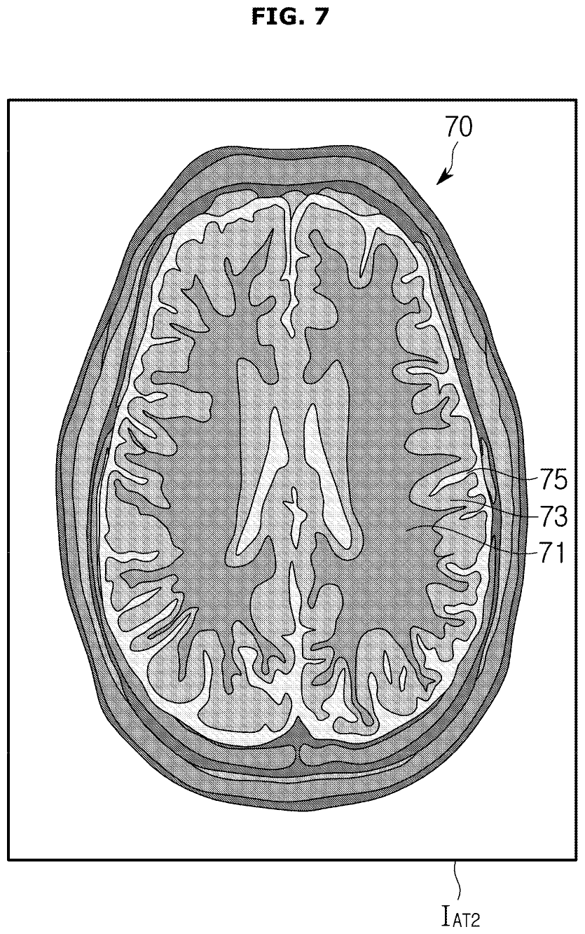

FIG. 6 is a graph illustrating the T2 decay curve of each configuration of the brain to explain a process of obtaining a T2-weighted image, and FIG. 7 is a view illustrating an example of the obtained T2-weighted image. In FIG. 6, an X-axis denotes an intensity of a signal and a Y-axis denotes a time.

As described above, when the RF coil 53 applies the RF pulse to the head of the subject 9 and then stops applying the RF pulse, the transverse magnetization vector of GM, WM and CSF existing in the head gradually decreases. In this case, the T2 decay curve CC3 is formed relatively smoothly because the CSF has a small out-phase, and the T2 decay curves CW3 and CG3 of each of WM and GM decrease relatively faster than the T2 decay curve CC3. The T2 decay curves CW3 and CG3 of WM and GM are also different from each other depending on the composition of WM and GM.

Therefore, when a signal at a certain time point, for example, after a predefined echo delay time or a lapse of the echo delay time, is measured, it becomes possible to obtain a T2-weighted image IAT2 in which the substances are displayed differently from each other, as illustrated in FIG. 6. In this case, since the signal output from the CSF is not suppressed, in the T2-weighted image IAT2, the part 75 where the CSF exists is brightly expressed and the part 71 where the WM exists is relatively darkened, and the part 73 where the GM exists is expressed by the brightness in the middle of the part 75 where the CSF exists and the part 71 where the WM exists.

Hereinafter, an exemplary embodiment of a process of obtaining the CSF image will be described.

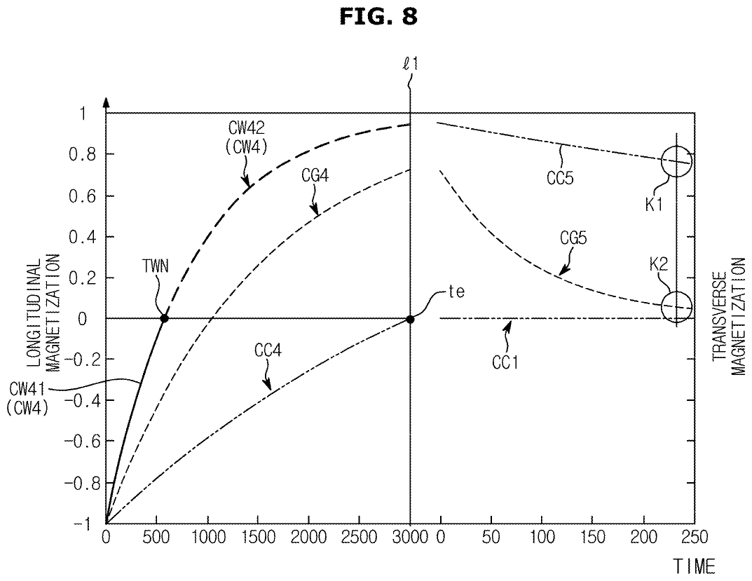

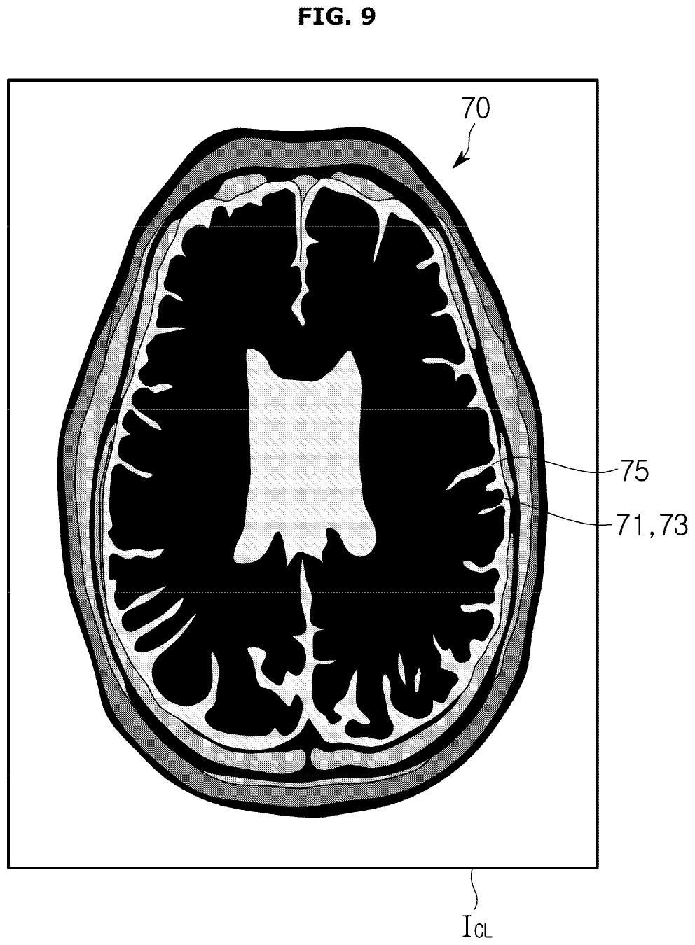

FIG. 8 is a view illustrating an inversion recovery curve and a T2 decay curve of each configuration of the brain to explain a process of obtaining a cerebrospinal fluid image, and FIG. 9 is a view illustrating an example of the obtained CSF image. As in the case of FIG. 4, in FIG. 8, the left side of vertical line (11) illustrates the time-dependent recovery of the longitudinal magnetization of each substance with the application of the inversion recovery pulse, and the right side of the vertical line (11) illustrates the time-dependent decay of the transverse magnetization.

When the inversion recovery pulse is applied to the head, the longitudinal magnetization of the atomic nucleus including each of WM, GM, and CSF is formed in the opposite direction and has a negative value. When the inversion recovery pulse is stopped, the longitudinal magnetization gradually recovers as time elapses, as illustrated in FIG. 8 (CW1, CG1, CC1).

According to an exemplary embodiment, when the longitudinal magnetization of WM reaches a first range while the atomic nucleus of each of WM, GM and CSF is recovered, by transmitting the excitation pulse to the atomic nucleus to excite the atomic nucleus, the electrical signal generated by WM is maintained at 0 or a very small value. Therefore, the longitudinal magnetization of WM is recovered to a certain point (CW41), but after the certain point, the longitudinal magnetization of WM is not recovered and maintains a value of 0 (CW42). Here, the first range may be defined to include only the case where the magnitude of the longitudinal magnetization of WM is 0. Also, the magnitude of the longitudinal magnetization of WM may be defined as a range including a value of 0 or a value close thereto (for example, 0+/−a). The first range may be defined and set by the user or designer.

When the magnitude of the longitudinal magnetization of WM is 0, only electrical signals generated in GM and CSF may be obtained.

Particularly, as illustrated on the right side of FIG. 8, the electrical signal due to the transverse magnetization of WM is hardly measured or ignored, and the transverse magnetization of GM and the transverse magnetization of CSF gradually become the T2 decay curve (CG5, CC5). In this case, the transverse magnetization of GM is relatively faster than the transverse magnetization of CSF, so that even if the magnitude of the transverse magnetization of GM reaches 0 or a value close thereto K2, the magnitude of the transverse magnetization of CSF is measured as a relatively large value K1. In this way, when the magnitude of the transverse magnetization of GM reaches 0 or a value close thereto K2, the signal obtained from the GM is 0 or has a very small value, and the signal obtained from the CSF is relatively larger than the signal obtained from the GM. Therefore, when the image is generated using the electrical signal at the time point when the magnitude of the transverse magnetization of GM reaches 0 or a value close thereto K2, as illustrated in FIG. 9, the GM may hardly appear, and the CSF may appear or the emphasized T2-weighted image may be obtained.

As described above, since the signal of the transverse magnetization of WM is not obtained by the excitation process and the signal of the transverse magnetization is also 0 or very small, the T2-weighted image obtained for the head 70, only the part 75 where the CSF exists is brightly expressed, and the part 71 where the WM exists and the part 73 where the GM exists are darkened. Thus, a CSF image ICL may be obtained.

According to another exemplary embodiment, when the longitudinal magnetization of GM reaches a first range while the atomic nucleus of each of WM, GM and CSF is recovered, by transmitting the excitation pulse to the atomic nucleus to excite the atomic nucleus as described above, the electrical signal generated by GM is maintained at 0 or a very small value. Here, the first range may be defined to include only the case where the magnitude of the longitudinal magnetization of GM is 0. Also, the magnitude of the longitudinal magnetization of GM may be defined as a range including a value of 0 or a value close thereto (for example, 0+/−a).

Subsequently, when the T2-weighted image is obtained at a time when the transverse magnetization of WM has a value close to 0 or 0, the part 71 where the WM exists and the part 73 where the GM exists appear dark, and the CSF image ICL in which only the part 75 where the CSF exists appears bright may be obtained.

Hereinafter, an example of a sequential process of obtaining the above-described CSF image ICL, flair image, and T2-weighted image according to the operation of the MRI apparatus 1 will be described.

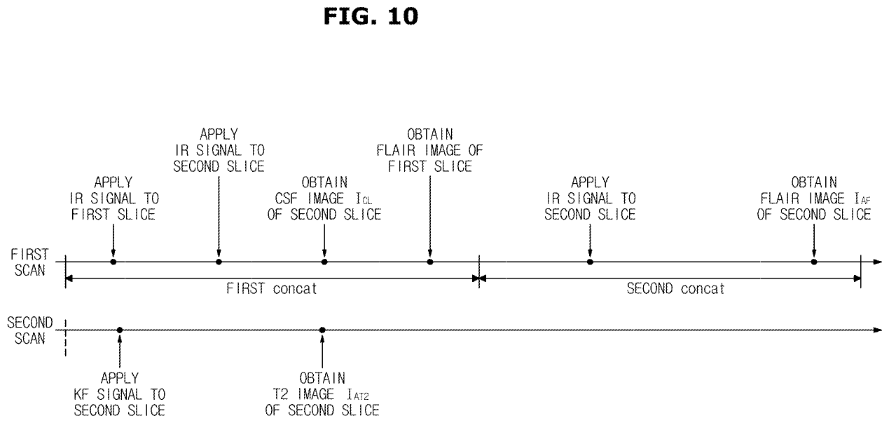

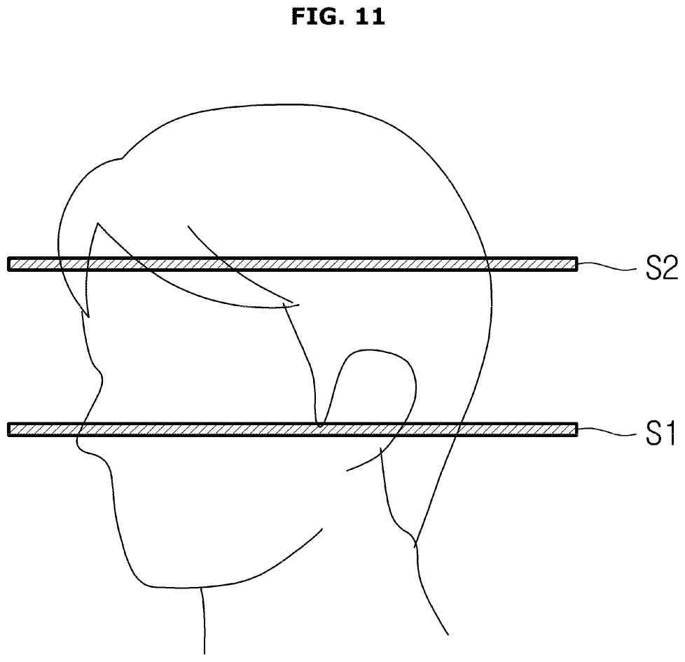

FIG. 10 is a view illustrating image acquisition in a first scan and a second scan, and FIG. 11 is a view illustrating a first slice and a second slice.

When the subject 9 is transferred to the internal space of the MRI apparatus 1 and the static magnetic field and/or the gradient magnetic field is applied to the subject 9, the RF signal is applied to the part to be measured, for example, each of the areas where the head is subdivided (hereinafter, referred to as ‘slice’). Here, the slice denotes a segment to be imaged, respectively.

In this case, as illustrated in FIG. 10, a second inversion recovery pulse may be applied first to a first slice s1 in a first concatenation of a first scan, and a first inversion recovery pulse may be applied to a second slice s2 after a predetermined time has elapsed. Here, the first slice s1 comprises a part of a nose of the human body as illustrated, for example, in FIG. 11, and the T2-weighted image may be closer to a parietal than the first slice s1, without being limited thereto.

When the second inversion recovery pulse is applied to the first slice s1, as illustrated in FIG. 4, the longitudinal magnetization of WM, GM, and CSF located in the first slice s1 is recovered after being reversed, and thus the recovery signal is received.

When the first inversion recovery pulse is applied to the second slice s2, the longitudinal magnetization of WM, GM, and CSF located in the second slice s2 is also recovered after being reversed.

When the longitudinal magnetization of WM located in the second slice s2 becomes 0 or approaches 0 (TWN) as illustrated in FIG. 8, the MRI apparatus 1 may excite the atomic nucleus in the second slice s2 so that the electrical signal generated by WM is 0 or has a very small value (WM Nulling). The MRI apparatus 1 may obtain the CSF image ICL at the second slice s2 using the electrical signal at the time point when the magnitude of the transverse magnetization of GM reaches 0 or a value close thereto K2. The CSF image ICL in the second slice s2 may be obtained earlier than the flair image because the excitation of the atomic nucleus is performed relatively quickly.

When the longitudinal magnetization of CSF in the first slice s1 reaches a value close to 0 or 0, the atomic nucleus is excited, and the T2-weighted image with the CSF removed based on the electrical signal corresponding to the longitudinal magnetization of WM and GM may be obtained. That is, the flair image for the first slice s1 may be obtained. The time point at which the longitudinal magnetization of CSF in the first slice s1 reaches a value close to 0 or 0 may be a time point during the acquisition of the CSF image ICL in the second slice s2, or may be a time point after the acquisition of the CSF image ICL in the second slice s2.

According to an exemplary embodiment, in the second concatenation of the first scan, the flair image IAF of the second slice s2 may be obtained. For example, in the second concatenation of the first scan, when the inversion recovery pulse is applied to the second slice s2 and the longitudinal magnetization of CSF reaches a value close to 0 or 0, the excitation may be performed to obtain the flair image IAF for the second slice s2. Accordingly, the CSF image ICL and the flair image IAF may be obtained together for one slice, for example, the second slice s2 in one scan.

In an exemplary embodiment, in the second concatenation of the first scan, the above-described first concatenation process may be repeated in the same manner, whereby a plurality of CSF images corresponding to each of a plurality of slices and a plurality of flair images corresponding to each of the plurality of slices may be obtained. In this case, the plurality of CSF images and the plurality of flair images corresponding to each of the plurality of slices may be obtained arbitrarily in order of the slices or irrespective of the order of the slices. For example, in the second concatenation, the CSF image in a third slice (not shown) that is different from the slice in which the CSF image is obtained in a previous concatenation, that is, the second slice s2, may be obtained. Also, in the previous concatenation, the flair image is obtained from a slice, for example, a slice different from the first slice s1, that is, the flair image in the second slice s2 from which the CSF image has been obtained in the previous concatenation, may be obtained.

More particularly, the CSF image for the third slice may be obtained in the same manner as described above between the time point when the inversion recovery pulse is applied to the second slice s2 and the time point when the flair image IAF for the second slice s2 is obtained. Particularly, the MRI apparatus 1 may apply the inversion recovery pulse to the second slice s2 and sequentially apply the inversion recovery pulse to the third slice. When the longitudinal magnetization of WM in the third slice becomes 0 or approaches 0, the atomic nucleus in the third slice may be excited, and the electrical signal at the time point when the magnitude of the transverse magnetization of GM reaches a value close to 0 or 0 may be measured to obtain the CSF image in the third slice.

According to this process, the plurality of flair images and CSF images corresponding to each slice may be obtained.

After the scanning of the MRI apparatus 1 ends once as described above, the MRI apparatus 1 may perform additional scanning, that is, a second scan, if necessary.

In the second scan, the MRI apparatus 1 may obtain only the T2-weighted image for each slice. For example, the MRI apparatus 1 may apply the RF pulse to the second slice s2 to obtain the T2-weighted image IAT2 for the second slice s2 as described with reference to FIGS. 6 and 7. Clearly, the MRI apparatus 1 may obtain the T2-weighted image for another slice, e.g., the first slice s1 or the third slice, in the second scan.

In the exemplary embodiment, the MRI apparatus 1 may employ an interleaved acquisition method when performing at least one of the first scan and the second scan.

In addition, FIG. 10 illustrates an example in which acquisition of the T2-weighted image for each slice is obtained by the second scan performed after the first scan, but acquisition of the T2-weighted image may also be obtained using a multiband radio frequency pulse method. In other words, acquisition of the T2-weighted image may be performed in the first scan process. For example, in the first concatenation process of the first scan, simultaneously with the process of applying the inversion recovery pulse to the first slice s1 and the second slice s2 for obtaining the flair image and the CSF image, the T2-weighted image in a fourth slice may be obtained by applying the RF pulse to another slice (hereinafter, referred to as ‘fourth slice’) different from the first slice s1 and the second slice s2. In other words, T2-weighted images may be generated in addition to flair images and CSF images.

In FIG. 10, the MRI apparatus 1 may obtain the CSF image, the flair image, and the T2-weighted image for each slice, for example, the second slice s2. However, according to the exemplary embodiment, the MRI apparatus 1 may not obtain at least one of the CSF image, the flair image, and the T2-weighted image. For example, the process of obtaining the flair image in the first scan process may be omitted. In this case, only the CSF image is obtained in the first scan. Also, for another example, the second scan may be omitted, in which case the T2-weighted image for each slice is not obtained.

The processor 30 of the MRI apparatus 1 may obtain at least one of the CSF image, the flair image, and the T2-weighted image through the process described above, and obtain a new image using the obtained image.

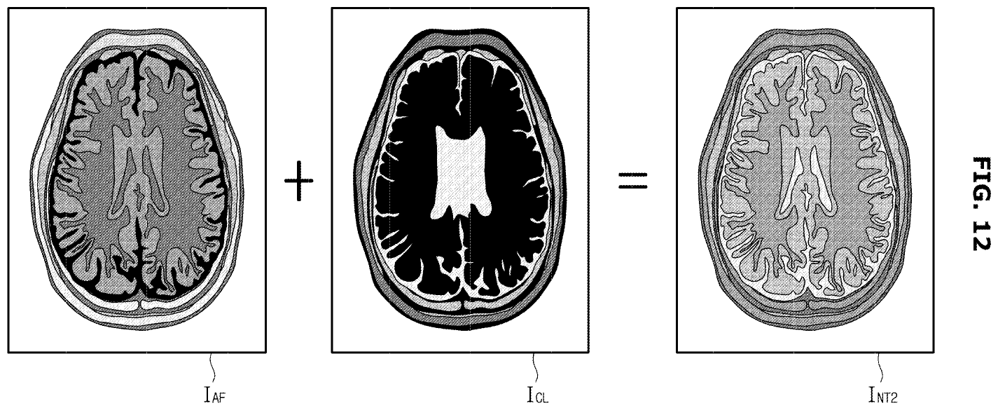

FIG. 12 is a view illustrating an example of obtaining a new T2-weighted image according to a combination of the flair image and a CSF-only image.

Referring to FIG. 12, the processor 30 may combine the flair image IAF and the CSF image ICL to obtain a new image. Since the flair image IAF is an image in which the CSF is not displayed and the CSF image ICL is an image in which the CSF-only image is displayed, when the flair image IAF and the CSF image ICL are combined, a new T2-weighted image INT2 may be obtained. The new T2-weighted image INT2 may not be substantially different from the T2-weighted image obtained directly by the MRI apparatus 1, for example, the T2-weighted image IAT2 obtained in the second scan. Accordingly, it is possible to acquire the T2-weighted image INT2 without performing the second scan, and when the second scan is performed, two identical or approximate T2-weighted images INT2 and IAT2 may be obtained.

The new T2-weighted image INT2 may be generated to some extent different from the T2-weighted image IAT2 directly obtained according to the combining method.

The processor 30 may use various image combining methods when combining the flair image IAF and the CSF image ICL. For example, the processor 30 may increase the transparency (alpha value) of each of the flair image IAF and the CSF image ICL, and generate an image by superimposing the flair image IAF and the CSF image ICL. Alternatively, in another example, the processor 30 may average image data of the respective pixels corresponding to each of the flair image IAF and the CSF image ICL, or may generate a new image by combining the flair image IAF and the CSF image ICL by taking an intermediate value of the image data of each pixel. In another example, the processor 30 may perform weighted summing, square summing, or complex summing of the flair image IAF and the CSF image ICL to obtain the new image.

When the second scan is performed, if two identical or approximate T2-weighted images INT2 and IAT2 are obtained, the processor 30 may further combine the two T2-weighted images INT2 and IAT2 (hereinafter, referred to as ‘final image’). In this case, the processor 30 may generate the final image by combining two T2-weighted images INT2 and IAT2 using the various image combining methods, for example, a method of obtaining an average image of the plurality of images, or weighted summing, square summing, or complex summing of the plurality of images. The final image may be the same as the T2-weighted image INT2 and IAT2, or may be partially different.

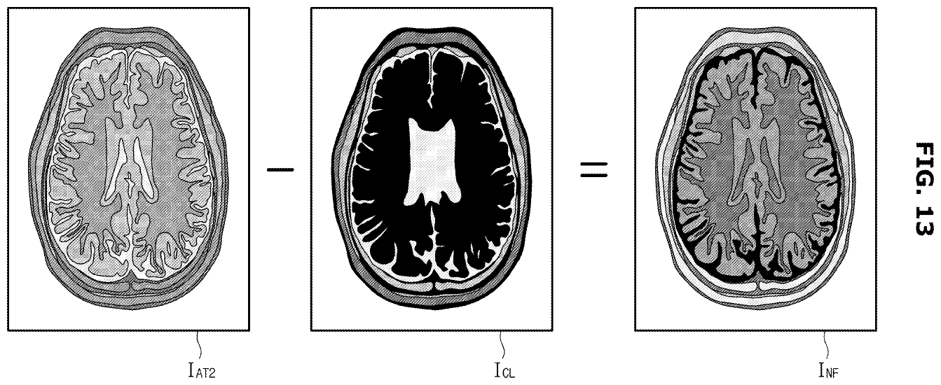

FIG. 13 is a view illustrating an example of obtaining a new flair image according to subtraction of the CSF-only image in the T2-weighted image.

Referring to FIG. 13, the processor 30 may obtain a new image using the T2-weighted image, for example, the T2-weighted image IAT2 obtained in the second scan and a CSF image ICL. For example, the processor 30 may obtain a new flair image INF (hereinafter, referred to as ‘first flair image’) for at least one first slice s1 and second slice s2 by subtracting the CSF image ICL from the T2-weighted image IAT2. As described above, since the CSF image ICL is the image in which the CSF-only image is displayed, when the CSF image ICL is subtracted from the T2-weighted image IAT2, the first flair image INF in which the CSF is not displayed may be obtained. The first flair image INF may be substantially the same as a flair image obtained directly by the MRI apparatus 1, for example, the flair image IAF (hereinafter, referred to as ‘second flair image’) for at least one first slice s1 and second slice s2 obtained in the first concatenation and/or the second concatenation of the first scan. Therefore, even if the flair image acquisition process is not performed in the first scan process, it is possible to obtain the flair image INF.

As illustrated in FIG. 10, when the first flair image is obtained for each slice, two identical or approximate flair images, i.e., the first flair image INF and the second flair image IAF, may be obtained. Here, as described above, the second flair image IAF may be obtained by causing the RF coil 53 to apply the inversion recovery pulse to the head, and suppressing the recovery signal generated in the CSF of the head.

As described above, the new first flair image INF may be different from the flair image IAF obtained in the first scan according to the method of subtracting the CSF image ICL from the T2-weighted image IAT2.

The processor 30 may subtract the CSF image ICL from the T2-weighted image IAT2 using various methods. For example, the processor 30 may obtain a new image by subtracting the value of the image data of each pixel of the corresponding CSF image ICL from the value of the image data of each pixel of the T2-weighted image IAT2. In this case, the processor 30 may add weight to either the value of the image data of each pixel of the T2-weighted image IAT2 and the value of the image data of each pixel of the CSF image ICL, and subtract the value of the image data of each pixel of the corresponding CSF image ICL from the value of the image data of each pixel of the T2-weighted image IAT2.

When two identical or approximate first flair image INF and second flair image IAF are obtained, the processor 30 may obtain a final image by combining two identical or approximate first flair image INF and second flair image IAF. In this case, the processor 30 may combine the two flair images IAF and INF using various image combining methods, for example, a method of averaging the plurality of flair images IAF and INF to obtain images, or weighted summing, square summing, or complex summing of the plurality of flair images IAF and INF. Thus, the final image, i.e., a new flair image, may be obtained.

As described above, the embodiment for obtaining the flair image, the CSF image, and/or the T2-weighted image from the head has been described. However, the disclosure is not limited to the example of scanning the head. For example, in the case of capturing a part of the subject 9 in which a signal of water is required to be suppressed, the above-described embodiment is equally or partially applicable. In this case, a component or substance image in which the signal of water is output in the area to be scanned may correspond to the CSF image.

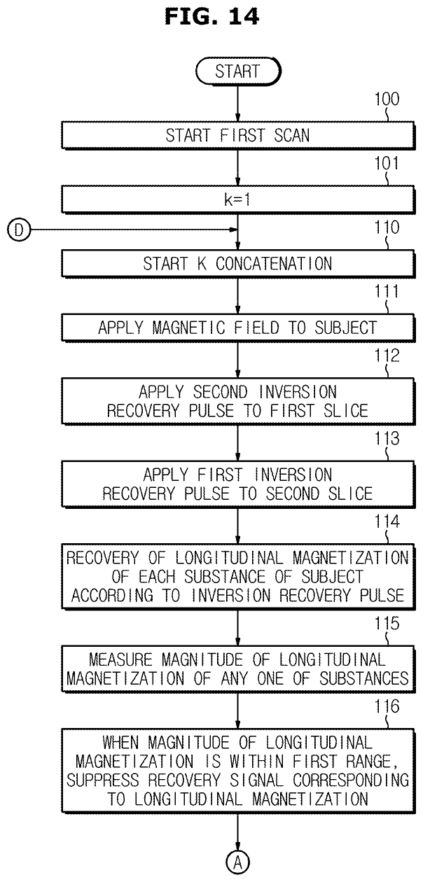

Hereinafter, an exemplary embodiment of a method of controlling the MRI apparatus will be described with reference to FIGS. 14 to 16.

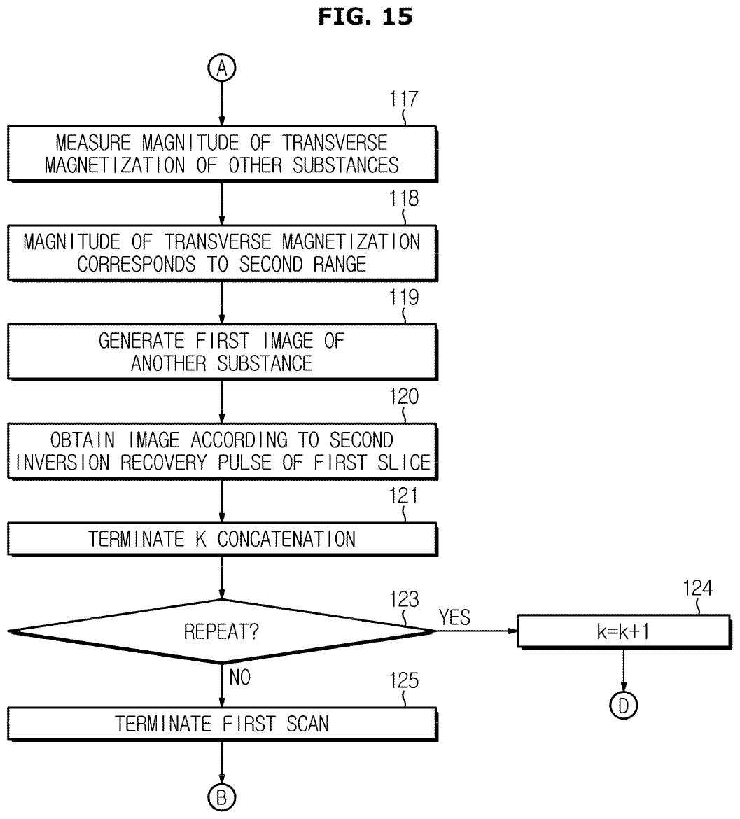

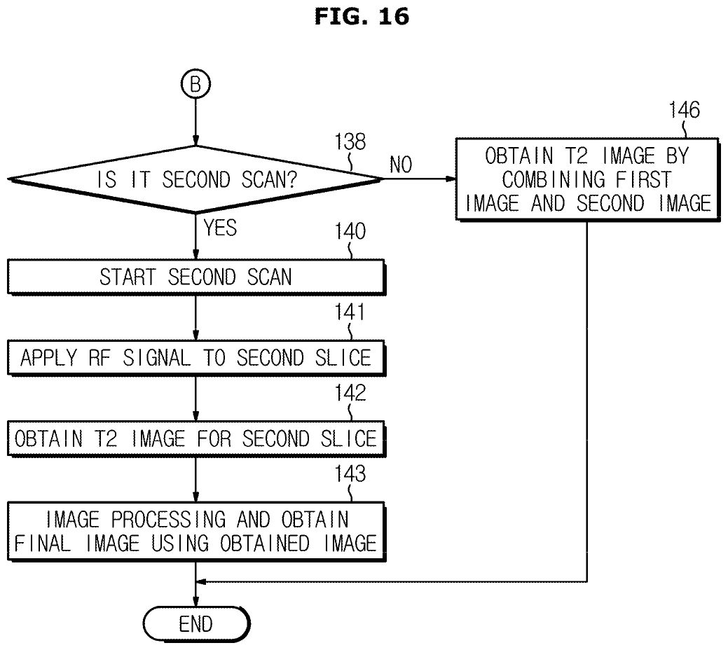

FIG. 14 is a first flowchart of a method of controlling the MRI apparatus according to an embodiment, FIG. 15 is a second flowchart of a method of controlling the MRI apparatus according to an embodiment, and FIG. 16 is a third flowchart of a method of controlling the MRI apparatus according to an embodiment.

Referring to FIG. 14, first, when the MRI apparatus 1 starts operating and the subject 9 enters the internal space, the first scan for the subject 9, for example, the head of the subject 9 is started (100). The operation according to the first concatenation with the start of the first scan is initiated (101, 110).

When the first concatenation is started, the magnetic field is applied to the subject 9 (111). Application of the magnetic field to the subject 9 may be performed before the start of the first concatenation (110).

The second inversion recovery pulse is applied to the first slice s1 of the subject 9 to which the magnetic field is applied (112). When the second inversion recovery pulse is applied, the longitudinal magnetization of each substance of the subject 9 (e.g., WM, GM and CSF) is formed in the opposite direction. When the application of the pulse is stopped, the longitudinal magnetization starts to be recovered.

The first inversion recovery pulse is applied to the second slice s2 of the subject 9 after the second inversion recovery pulse is applied and immediately or after the certain time has elapsed (113). Here, the second slice s2 is located at a different position between the first slice s1 and the subject 9.

The longitudinal magnetization of each substance of the subject 9 existing in the second slice s2, for example, WM, GM and CSF is formed in the opposite direction as described above according to the application of the first inversion recovery pulse for the second slice s2, and the longitudinal magnetization of each substance of the subject 9 begins to recover as the application of the first inversion recovery pulse is terminated (114).

The MRI apparatus 1 may measure the magnitude of the longitudinal magnetization of any one of the substances in the second slice s2, for example, WM and GM (115). In this case, the measurement of the longitudinal magnetization may be performed based on the magnitude of the electrical signal output.

When the magnitude of the longitudinal magnetization of any one of the substances of WM and GM, for example, corresponds to the first range, the excitation pulse is applied to the subject 9, so that at least one recovery signal of any one of the substances of WM and GM is suppressed (116). Here, the first range denotes a range in which the magnitude of the longitudinal magnetization of any one of the substances is set to 0 or a value close thereto. The first range may be defined to include only a value of 0.

Referring to FIG. 15, the MRI apparatus 1 may measure the magnitude of another transverse magnetization in which the recovery signal of the other substance in the second slice s2, for example, WM and GM, is not suppressed (117). The transverse magnetization of each substance generated due to the RF pulse is reduced with time when the RF pulse application is stopped.

When the magnitude of the transverse magnetization, which is decreased with time, corresponds to a second range (118), a first image for another of the respective substances may be generated based on the electrical signal at the time point when the magnitude of the transverse magnetization corresponds to the second range (119). Thus, a CSF image ICSF for the second slice s2 may be obtained. Here, the second range may be defined as a range in which the magnitude of the transverse magnetization of the other one of the substances, e.g., WM and GM, where the recovery signal is not suppressed, is 0, or a range that includes a value close to 0 and 0.

A second image, e.g., a flair image, of the first slice s1 according to the second inversion recovery pulse applied to the first slice s1 may be obtained (120). When the magnitude of the longitudinal magnetization of CSF corresponds to a third range, for example, 0 or a value close thereto, for the acquisition of the flair image, the MRI apparatus 1 may apply an excitation pulse to each substance of the first slice s1 so that the atomic nucleus of each substance can be applied. As a result, the electrical signal for another substance of the first slice s1, for example, the CSF, may be suppressed and the image corresponding to the second inversion recovery pulse for the first slice s1, that is, the flair image, may be obtained. Here, the application of the excitation pulse may be performed at any time point in the operation for applying the first inversion recovery pulse or the operation for obtaining the first image (113 to 119), or may be performed after the acquisition of the first image (119) according to another substance.

In this way, the MRI apparatus 1 may obtain two different types of images, for example, the CSF image and the flair image.

When the second image for the first slice s1 and the first image for the second slice s2 are obtained, the first concatenation is terminated (121).

Steps 110 to 123 for obtaining the plurality of images in one concatenation may be sequentially repeated (123). When the second concatenation, the third concatenation, and the like are sequentially performed (YES in 123), as illustrated in FIG. 15, the acquisition process of the first image and the second image for each of the concatenations (110 to 123) are sequentially repeated (124). In this case, in a new concatenation, the first image in the slice different from the slice in which the first image is generated in the existing concatenation, for example, the CSF image in the different slice, may be obtained, and the second image in the slice different from the slice in which the second image is generated in the existing concatenation, for example, the flair image in the different slice, may be obtained.

For example, in the second concatenation, the second image at the second slice s2, e.g., the flair image, may be obtained, and the first image at the third slice, e.g., the CSF image, may be obtained. In the third concatenation, the second image at the third slice may be obtained, and the first image at the fourth slice may be obtained.

Through the above process, the MRI apparatus 1 may obtain a plurality of first images and a plurality of second images for each of the plurality of slices corresponding to a plurality of concatenations.

When a last concatenation is terminated, no further repetition is necessary (NO in 123), and thus the first scan of the MRI apparatus 1 is terminated (125).

Step 112 of applying the second inversion recovery pulse to the first slice s1 and operation 120 of obtaining the image according to the second inversion recovery pulse of the first slice s1 in the first scan described above (100 to 125) may be omitted according to the embodiment.

As illustrated in FIG. 16, when the first scan is terminated, the second scan may be further performed, if necessary (138).

When the second scan is not performed (NO in 138), the MRI apparatus 1 may obtain the new image using two types of images obtained according to the first scan, i.e., at least one first image and at least one second image (146). When the first image is the CSF image and the second image is the flair image, the image obtained according to the combination of the first image and the second image may be the same as or nearly similar to the T2-weighted image. Therefore, even if the second scanning process (138 to 142) is not performed, the MRI apparatus 1 may obtain the T2-weighted image.

According to an exemplary embodiment, the MRI apparatus 1 may generate a new image that is the same as the T2-weighted image, approximately the T2-weighted image, or is different from the T2-weighted image by obtaining the average image between the first image and the second image or by overlapping the first image and the second image, or by performing weighted summing, square summing, or complex summing of the image data of the pixels corresponding to the first image and second image.

When the second scan is set to be performed (YES in 138), the MRI apparatus 1 starts the second scan for the subject 9 (150).

The MRI apparatus 1 may apply the RF pulse to at least one slice, e.g., the second slice s2 (151), and generate and obtain the third image, for example, the T2-weighted image, based on the magnetic resonance signal received from the substance located at the second slice s2 (152). Here, the slice to which the RF pulse is applied may be a slice in which the first image is obtained in the first scan process (100 to 125). The acquisition of T2-weighted images may be performed using the multiband radio frequency pulse method according to the embodiment.

The MRI apparatus 1 may obtain at least one new image based on at least one of the first image, the second image, and the third image, and perform image processing on the first image, the second image, the third image, and obtain at least one new image (143).

For example, when the first image is the CSF image and the third image is the T2-weighted image, the MRI apparatus 1 may obtain the new image by subtracting the first image from the third image, and the obtained new image is the same as or approximate to the second image, i.e., the flair image, obtained by the second inversion recovery signal. Accordingly, even if operations 112 and 120 for obtaining the second image are omitted, the MRI apparatus 1 may obtain the flair images.

In addition, when the first image is the CSF image and the second image is the flair image, the MRI apparatus 1 may obtain the new image by combining the first image and the second image. In this case, the new image is the same as or approximate to the T2-weighted image.

The MRI apparatus 1 may obtain the final image by combining the new image obtained through image processing and another image obtained directly corresponding to the new image.

For example, when the new image, for example, the T2-weighted image in operation 143 is generated, the MRI apparatus 1 may obtain another new image using at least one of a method of combining the new T2-weighted image and the third image obtained in the second scan process 138 to 142, that is, the T2-weighted image, a method of subtracting the T2-weighted image obtained in the second scan process 138 to 142 from the new T2-weighted image, or various other image processing methods.

In another example, when the new image, that is, the flair image is obtained by subtracting the CSF image (the second image) from the T2-weighted image (the third image) in operation 143, the MRI apparatus 1 may obtain another new image using a method of combining the second image obtained through operation 112 and operation 120, that is, the flair image, and the flair image obtained in operation 143, or the like.

The method of controlling the MRI apparatus according to the above-described embodiment may be implemented in the form of a program that can be executed by various computer devices. The program may include program instructions, data files, and data structures alone or in combination. The program may be designed or manufactured by using a machine language code or a complex language code. In addition, the program may be particularly designed to implement the above-described methods or may be implemented by using various functions or definitions that are well-known and available to those of ordinary skill in the computer software field.

A program for implementing the method of controlling the MRI apparatus may be recorded on a computer-readable recording medium. The computer-readable recording medium may include various types of hardware devices capable of storing specific programs that are executed in response to a call from a computer, e.g., magnetic disk storage media such as a hard disk or a floppy disk; optical recording media such as a magnetic tape, a compact disc (CD) or a DVD; magneto-optical recording media such as a floptical disk; and semiconductor memory devices such as ROM, RAM, or flash memory.

Hereinbefore, various embodiments of the MRI apparatus and the method of controlling the MRI apparatus have been described. However, the apparatus and the method are not limited to the above-described embodiments. Various apparatuses or methods that can be implemented by one of ordinary skill in the related art through correction and modification based on the above-described embodiments may also be examples of the above-described apparatus and method for defending the unauthorized modification of the program. For example, although the above-described techniques are performed in a different order from that of the above-described method, and/or the above-described components, such as systems, structures, apparatuses, and circuits, and are coupled or combined in a different form from that of the above-described method, or replaced or substituted with other components or equivalents, proper results can be achieved.

1. A magnetic resonance imaging (MRI) apparatus comprising:
a magnetic field generator configured to apply a magnetic field to a head of a subject;
a radio frequency (RF) coil configured to apply a pulse to the head to which the magnetic field is applied, and to receive a signal generated in the head; and
a processor configured to:
apply a first inversion recovery pulse to the head by the RF coil,
when a magnitude of longitudinal magnetization of one of white matter (WM) and gray matter (GM) of the head is in a first range, suppress a recovery signal corresponding to the longitudinal magnetization of at least one of the WM and GM among recovery signals generated according to the first inversion recovery pulse; and
generate a cerebrospinal fluid (CSF) image for at least one slice based on a signal at a point where a magnitude of transverse magnetization generated in the other of the WM and GM of the head is in a second range.

2. The MRI apparatus according to claim 1, wherein when the RF coil applies a second inversion recovery pulse to the head and a magnitude of longitudinal magnetization of the CSF of the head is in a third range, the processor is configured to suppress a recovery signal generated in the CSF according to the second inversion recovery pulse to obtain a flair image for a slice different from the at least one slice.

3. The MRI apparatus according to claim 2, wherein the RF coil is configured to apply the second inversion recovery pulse to the head, and to apply the first inversion recovery pulse to the head before the flair image for the slice different from the at least one slice is obtained.

4. The MRI apparatus according to claim 1, wherein the RF coil is configured to apply the pulse to the head, and
wherein the processor is configured to generate a second T2-weighted image for the at least one slice based on a magnetic resonance signal received by the RF coil.

5. The MRI apparatus according to claim 4, wherein the RF coil is configured to apply an inversion recovery pulse to the head, and
wherein the processor is configured to suppress a recovery signal generated in the CSF of the head to obtain a first flair image for the at least one slice.

6. The MRI apparatus according to claim 5, wherein the processor is configured to combine the first flair image and the CSF image for the at least one slice to obtain a first T2-weighted image for the at least one slice.

7. The MRI apparatus according to claim 6, wherein the processor is configured to combine the first T2-weighted image and the second T2-weighted image by performing weighted summing, square summing, or complex summing of the first T2-weighted image and the second T2-weighted image to obtain a final image.

8. The MRI apparatus according to claim 4, wherein the processor is configured to obtain a second flair image for the slice by subtracting the CSF image from the second T2-weighted image.

9. The MRI apparatus according to claim 8, wherein the processor is configured to obtain a final image based on a first flair image and the second flair image.

10. The MRI apparatus according to claim 4, wherein the processor is configured to obtain the magnetic resonance signal using a multiband radio frequency pulse method or to obtain the magnetic resonance signal using an interleaved acquisition method.

11. The MRI apparatus according to claim 1, wherein the first range comprises the magnitude of the longitudinal magnetization of the one of the WM and GM of the head at a value of 0 or a value close to 0, and
wherein the second range comprises the magnitude of the transverse magnetization of the other of the WM and GM of the head at a value of 0 or [symbol] value close to 0.

12. A method of controlling a magnetic resonance imaging (MRI) apparatus comprising:
applying a magnetic field to a head of a subject;
applying a first inversion recovery pulse to the head to which the magnetic field is applied,
when a magnitude of longitudinal magnetization of one of white matter (WM) and gray matter (GM) of the head is in a first range, suppressing a recovery signal corresponding to the longitudinal magnetization of at least one of the WM and GM among recovery signals generated according to the first inversion recovery pulse; and
generating a cerebrospinal fluid (CSF) image for at least one slice based on a signal at a point where a magnitude of transverse magnetization generated in the other of the WM and GM of the head is in a second range.

13. The method according to claim 12, further comprising:
applying a second inversion recovery pulse to the head to which the magnetic field is applied; and
when a magnitude of longitudinal magnetization of the CSF of the head is in a third range, suppressing a recovery signal generated in the CSF according to the second inversion recovery pulse to obtain a flair image for a slice different from the at least one slice.

14. The method according to claim 13, wherein the applying of the first inversion recovery pulse to the head to which the magnetic field is applied is performed after the applying of the second inversion recovery pulse to the head to which the magnetic field is applied.

15. The method according to claim 12, further comprising:
obtaining a flair image for the at least one slice; and
combining the flair image for the at least one slice and the CSF image for the at least one slice to obtain a first T2-weighted image for the at least one slice.

16. The method according to claim 15, further comprising:
receiving a magnetic resonance signal generated in the head;
generating a second T2-weighted image for the at least one slice based on the magnetic resonance signal; and
obtaining a final image based on the first T2-weighted image and the second T2-weighted image.

17. The method according to claim 16, wherein the obtaining of the final image based on the first T2-weighted image and the second T2-weighted image comprises:
combining the first T2-weighted image and the second T2-weighted image by performing weighted summing, square summing, or complex summing of the first T2-weighted image and the second T2-weighted image to obtain the final image.

18. The method according to claim 12, further comprising:
receiving a magnetic resonance signal generated in the head;
generating a T2-weighted image for the at least one slice based on the magnetic resonance signal; and
obtaining a second flair image for the at least one slice by subtracting the CSF image from the T2-weighted image.

19. The method according to claim 18, further comprising:
applying an inversion recovery pulse to the head to which the magnetic field is applied;
suppressing a recovery signal generated in the CSF of the head to obtain a first flair image for the at least one slice; and
obtaining the final image based on the first flair image and the second flair image.

20. The method according to claim 12, wherein the first range comprises the magnitude of the longitudinal magnetization of either the WM or GM of the head at a value of 0 or a value close to 0, and
wherein the second range comprises the magnitude of the transverse magnetization of the other of the WM and GM of the head at a value of 0 or a value close to 0.
